(19) United States
(12) Patent Application Publication
(10) Pub. No.: US 2008/0226734 A1
(43) Pub. Date: Sep. 18, 2008

(54) COMBINATION OF A NARCOTIC AND NON-NARCOTIC ANALGESIC

(75) Inventors: Scott Jenkins, Downington, PA (US); Gary Liversidge, Westchester, PA (US)

Correspondence Address: Fox Rothschild, LLP;Elan Pharma International Limited, 2000 Market Street, Philadelphia, PA, 19103, US

(73) Assignee: ELAN CORPORATION PLC, Dublin (IE)

(21) Appl. No.: 12/049,046

(22) Filed: Mar. 14, 2008

Related U.S. Application Data
(60) Provisional application No. 60/895,155, filed on Mar. 16, 2007.

(51) Int. Cl. A61K 9/14; A61P 29/00

(52) U.S. Cl. 424/489

(57) ABSTRACT

The present invention is directed to a formulation comprising a narcotic analgesic and a non-narcotic analgesic, methods of use and methods of preparing thereof.

### CROSS REFERENCE TO RELATED APPLICATIONS

This application claims priority under 35 U.S.C. § 119(e) to U.S. Provisional Application Ser. No. 60/895,155, which was filed on Mar. 16, 2007, the disclosure of which is incorporated herein by reference.

### FIELD OF THE INVENTION

The present invention relates to a formulation useful for delivery of a narcotic and a non-narcotic analgesic.

### SUMMARY OF THE INVENTION

In accordance with this invention, there is provided a formulation comprising: (A) a narcotic analgesic; and (B) a non-narcotic analgesic.

Another aspect of the present invention is a formulation comprising a narcotic analgesic and a non-narcotic analgesic in which the narcotic analgesic and the non-narcotic analgesic are released such that the duration of action of the narcotic analgesic matches that of the non-narcotic analgesic.

Yet another aspect of the present invention is the provision of a method for the treatment of pain comprising the step of delivering to a patient a formulation comprising a narcotic analgesic and a non-narcotic analgesic.

A further aspect of the present invention is the provision of a method for preparing a formulation which is useful in the treatment of pain comprising the step of mixing a narcotic analgesic and a non-narcotic analgesic.

### DETAILED DESCRIPTION OF THE INVENTION

The formulation of the present invention comprises a narcotic analgesic and a non-narcotic analgesic. For the purpose of the present application, the term “narcotic analgesic” includes precursors, congeners, salts, complexes, analogs, and derivatives of a narcotic analgesic and the term “non-narcotic analgesic” includes precursors, congeners, salts, complexes, analogs, and derivatives of a non-narcotic analgesic.

The narcotic analgesic is present in the composition in a pharmaceutically-effective amount. “Pharmaceutically-effective amount”, as used in the present application with respect to an active compound, means that the compound is present in an amount that allows for the specific pharmacological response for which the compound is administered to be exhibited in a significant number of subjects that are in need of such treatment. It is understood that, even though a certain amount may be deemed a “pharmaceutically-effective amount”, it may be the case that, when administered to a specific subject in a specific instance, the desired pharmacological response may not be obtained.

For guideline purposes, it is believed most applications will involve the use of a narcotic analgesic in an amount of about 0.5 mg to about 1000 mg, about 0.5 mg to about 800 mg, about 1 mg to about 600 mg, 1 mg to about 200 mg, about 1 mg to about 150 mg, or about 1 mg to about 100 mg.

Examples of narcotic analgesics that may be used in the practice of the present invention include oxycodone, oxymorphone, codeine, morphine, hydromorphone, levorphanol, methadone, meperidine, butorphanol, alfentanil, sufentanil, fentanyl, propoxyphene, levomethadyl, remifentanil, tramadol and hydrocodone.

The non-narcotic analgesic is present in the composition in a pharmaceutically-effective amount. For guideline purposes, it is believed most applications will involve the use of the non-narcotic analgesic in an amount of about 0.5 mg to about 1000 mg, about 0.5 mg to about 800 mg, about 1 mg to about 600 mg, 1 mg to about 200 mg, about 1 mg to about 150 mg, or about 1 mg to about 100 mg.

Examples of non-narcotic analgesics that may be used in the practice of the present invention include aspirin, ibuprofen, acetaminophen, NSAIDs, and COXII drugs.

In an embodiment of the present invention, at least one of the active compounds (the narcotic analgesic or the non-narcotic analgesic) is contained in a nanoparticle. A formulation is said to be a “nanoparticulate” formulation if the particles therein have an effective average particle size of less about 2000 rim, as measured by appropriate methods, for example, sedimentation flow fractionation, photon correlation spectroscopy, light scattering methods, disk centrifugation, or other techniques known to those of skill in the art. “Effective average particle size” refers to the average particle size of the particles in the formulation. The individual particles are known as “nanoparticles”. The nanoparticle comprises the active compound and a surface modifier. The surface modifier is associated with the surface of the nanoparticle and prevents the nanoparticle from agglomerating with other nanoparticles. More than one surface modifier may be used.

It is known in the art that a drug contained in a nanoparticulate dosage form exhibits improved bioavailability as compared with the same drug in a non-nanoparticulate dosage form. This is because the rate of the dissolution of a drug contained in a dosage form is increased when the surface area of the dosage form is increased. A nanoparticulate dosage form has a relatively large surface area and thus exhibits improved dissolution for the drag contained therein.

In an embodiment of the present invention, the nanoparticles in the formulation have an effective average particle size of less about 2000 nm, as measured by methods such as those described above. In various other embodiments of the present invention, the nanoparticles have an effective average particle size of less than about 1900 nm, about 1800 nm, about 1700 nm, about 1600 nm, about 1500 nm, about 1400 nm, about 1300 nm, about 1200 nm, about 1100 nm, about 1000 nm, about 900 nm, about 800 nm, about 700 nm, about 600 nm, about 500 nm, about 400 nm, about 300 nm, about 250 nm, about 200 nm, about 150 nm, about 100 nm, about 75 nm, or about 50 nm, as measured by appropriate methods such as those described above.

As another form of measurement, “D50”, when used with reference to a particle size refers to the size below which 50% of the particles fall, as measured using methods such as the above. Likewise, “D90”, when used with reference to a particle size refers to the size below which 90% of the particles fall, as measured using methods such as the above.

The surface modifier used must be specifically one which is capable of preventing the agglomeration of nanoparticles which contain the specific active compound of interest with other nanoparticles. Essentially any surface modifier capable of associating with the surface of a nanoparticle containing the active compound of interest (a narcotic analgesic or a non-narcotic analgesic) and preventing it from agglomerating with another nanoparticle may be used in the practice of the present invention. Examples of suitable surface modifiers include gelatin, casein, lecithin, gum acacia, cholesterol, tragacanth, stearic acid, benzalkonium chloride, calcium stearate, glyceryl monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters, polyethylene glycols, polyoxyethylene stearates, colloidal silicon dioxide, phosphates, sodium dodecylsulfate, carboxymethylcellulose calcium, carboxymethylcellulose sodium, methylcellulose, hydroxyethylcellulose, hydroxypropylcellulose, hydroxypropylmethylcellulose phthalate, noncrystalline cellulose, magnesium aluminum silicate, triethanolamine, polyvinyl alcohol, polyvinylpyrrolidone, an ethylene oxide-propylene oxide block copolymer (e.g., poloxamers), dioctylsulfosuccinate, sodium lauryl sulfate, dextran, 4-(1,1,3,3-tetramethylbutyl)-phenol polymer with ethylene oxide and formaldehyde, poloxamines, alkyl aryl polyether sulfonates, mixtures of sucrose stearate and sucrose distearate, p-isononylphenoxypoly-(glycidol), glucamides, glucopuranosides, maltosides, glucosides, PEG-phospholipid, PEG-cholesterol, PEG-cholesterol derivative, PEG-vitamin A, PEG-vitamin E, lysozyme, random copolymers of vinyl pyrrolidone and vinyl acetate, polymers, biopolymers, polysaccharides, cellulosics, alginates, phospholipids, zwitterionic stabilizers, pyridinum compounds, oxonium compounds, halonium compounds, cationic organometallic compounds, quarternary phosphorous compounds, anilinium compounds, ammonium compounds, chitosan, polylysine, polyvinylimidazole, polybrene, polymethylmethacrylate trimethylammoniumbromide bromide (PMMTMABr), hexyldesyltrimethylammonium bromide (HDMAB), polyvinylpyrrolidone-2-dimethylaminoethyl methacrylate dimethyl sulfate, cationic lipids, sulfonium, phosphonium, choline esters, stearalkonium chloride compounds, cetyl pyridinium bromide or chloride, halide salts of quaternized polyoxyethylalkylamines, alkyl pyridinium salts, amines, amine salts, imide azolinium salts, protonated quaternary acrylamides, methylated quaternary polymers, cationic guar, and a carbonium compound.

In embodiments in which the surface modifier is an ammonium compound, the modifier may be a primary ammonium compound, a secondary ammonium compound, a tertiary ammonium compound, or a quarternary ammonium compound. The quarternary ammonium compound may be one of the formula NR₁R₂R₃R₄⁽⁺⁾ in which:
(i) none of R₁-R₄ is CH₃;
(ii) one of R₁-R₄ is CH₃;
(iii) three of R₁-R₄ are CH₃;
(iv) all of R₁-R₄ are CH₃;
(v) two of R₁-R₄ are CH₃, one of R₁-R₄ is C₆H₅CH₂, and one of R₁-R₄ is an alkyl chain of seven carbon atoms or less;
(vi) two of R₁-R₄ are CH₃, one of R₁-R₄ is C₆H₅CH₂, and one of R₁-R₄ is an alkyl chain of nineteen carbon atoms or more;
(vii) two of R₁-R₄ are CH₃ and one of R₁-R₄ is the group C₆H₅(CH₂)ₙ, where n>1;
(viii) two of R₁-R₄ are CH₃, one of R₁-R₄ is C₆H₅CH₂, and one of R₁-R₄ comprises at least one heteroatom;
(ix) two of R₁-R₄ are CH₃, one of R₁-R₄ is C₆H₅CH₂, and one of R₁-R₄ comprises at least one halogen;
(x) two of R₁-R₄ are CH₃, one of R₁-R₄ is C₆H₅CH₂, and one of R₁-R₄ comprises at least one cyclic fragment;
(xi) two of R₁-R₄ are CH₃ and one of R₁-R₄ is a phenyl ring; or
(xii) two of R₁-R₄ are CH₃ and two of R₁-R₄ are purely aliphatic fragments.

Examples of such modifiers include, but are not limited to, behenalkonium chloride, benzethonium chloride, cetylpyridinium chloride, behentrimonium chloride, lauralkonium chloride, cetalkonium chloride, cetrimonium bromide, cetrimonium chloride, cethylamine hydrofluoride, chlorallylmethenamine chloride (Quaternium-15), distearyldimonium chloride (Quaternium-5), dodecyl dimethyl ethylbenzyl ammonium chloride (Quaternium-14), Quaternium-22, Quaternium-26, Quaternium-18 hectorite, dimethylaminoethylchloride hydrochloride, cysteine hydrochloride, diethanolammonium POE (10) oletyl ether phosphate, diethanolammonium POE (3) oleyl ether phosphate, tallow alkonium chloride, dimethyl dioctadecylammoniumbentonite, stearalkonium chloride, domiphen bromide, denatonium benzoate, myristalkonium chloride, laurtrimonium chloride, ethylenediamine dihydrochloride, guanidine hydrochloride, pyridoxine HCl, iofetamine hydrochloride, meglumine hydrochloride, methylbenzethonium chloride, myrtrimonium bromide, oleyltrimonium chloride, polyquatemium-1, procainehydrochloride, cocobetaine, stearalkonium bentonite, stearalkoniumhectonite, stearyl trihydroxyethyl propylenediamine dihydrofluoride, tallowtrimonium chloride, and hexadecyltrimethyl ammonium bromide.

The surface modifiers are commercially available and/or can be prepared by techniques known in the art. Most of these surface modifiers are known pharmaceutical excipients and are described in detail in the Handbook of Pharmaceutical Excipients, published jointly by the American Pharmaceutical-Association and The Pharmaceutical Society of Great Britain (The Pharmaceutical Press, 2000).

The relative amounts of the active compound and surface modifier within the nanoparticle can vary widely. The optimal amount of the individual components can depend, for example, upon the particular active compound selected, the hydrophilic lipophilic balance (HLB), melting point, and the surface tension of water solutions of the modifier. The concentration of the active compound within the nanoparticle can vary from about 99.5% to about 0.001%, from about 95% to about 0.1%, or from about 90% to about 0.5%, by weight, based on the total combined dry weight of the active compound and the surface modifier, not including other excipients. The concentration of the surface modifier can vary from about 0.5% to about 99.999%, from about 5.0% to about 99.9%, or from about 10% to about 99.5%, by weight, based on the total combined dry weight of the NSAID and surface modifier, not including other excipients.

In an embodiment of the present invention, the surface modifier is adsorbed onto the surface.

In various embodiments of the present invention, the nanoparticle may be in the form of a crystal (hereafter, a “nanocrystal”), a pellet, a bead, a granule, or a sphere.

In an embodiment of the present invention, the formulation contains nanoparticles which comprise an active compound and exhibits, when assayed in the plasma of a mammalian subject: a Cₘₐₓ for the active compound that is greater than the Cₘₐₓ for the same active compound when administered at the same dosage but in a non-nanoparticulate form; an AUC for the active compound that is greater than the AUC for the same active compound when administered at the same dosage but in a non-nanoparticulate form; and/or a Tₘₐₓ for the active compound that is less than the Tₘₐₓ for the same active compound when administered at the same dosage but in a non-nanoparticulate form. In various embodiments of the present invention, the formulation may exhibit a Cₘₐₓ for the active compound that is at least about 50%, about 100%, about 200%, about 300%, about 400%, about 500%, about 600%, about 700%, about 800%, about 900%, about 1000%, about 1100%, about 1200%, about 1300%, about 1400%, about 1500%, about 1600%, about 1700%, about 1800%, or about 1900% greater than the Cₘₐₓ for the same active compound when administered at the same dosage but in a non-nanoparticulate form. In various embodiments of the present invention, the formulation may exhibit an AUC for the active compound that is at least about 25%, about 50%, about 100%, about 125%, about 150%, about 175%, about 200%, about 225%, about 250%, about 275%, about 300%, about 350%, about 400%, about 450%, about 500%, about 550%, about 600%, about 700%, about 750%, about 800%, about 850%, about 900%, about 950%, about 1000%, about 1050%, about 1100%, about 1150%, or about 1200% greater than the AUC for the same active compound when administered at the same dosage but in a non-nanoparticulate form. In various embodiments of the present invention, the formulation may exhibit a Tₘₐₓ for the active compound that is not greater than about 90%, about 80%, about 70%, about 60%, about 50%, about 30%, about 25%, about 20%, about 15%, about 10%, or about 5% of the Tₘₐₓ for the same active compound when administered at the same dosage but in a non-nanoparticulate form.

In an embodiment of the invention, the active compound is contained in nanoparticles and the Tₘₐₓ for the active compound, when assayed in the plasma of a mammalian subject, is less than about 6 to about 8 hours after administration. In various other embodiments of the invention, the active compound is contained in nanoparticles and the Tₘₐₓ for the active compound, when assayed in the plasma of a mammalian subject, is less than about 6 hours, about 5 hours, about 4 hours, about 3 hours, about 2 hours, about 1 hour, or about 30 minutes after administration.

In an embodiment of the present invention, the active compound is contained in nanoparticles and there is no substantial difference in the quantity of the active compound absorbed or the rate of drug absorption when the formulation containing the nanoparticles is administered in the fed state versus the fasted state. The benefit of such an embodiment is that it substantially eliminates the effect of food and, thereby, increases patient compliance as the subject no longer needs to take a dose of the formulation with or without food. In various embodiments of the present invention, the difference in AUC or Cmax of the NSAID when administered in the fed versus the fasted state is less than about 60%, about 55%, about 50%, about 45%, about 40%, about 35%, about 30%, about 25%, about 20%, about 15%, about 10%, about 5%, or about 3%. In one embodiment, the active compound is contained in nanoparticles and the administration of the active compound in the fed state is bioequivalent to the administration of the active compound in the fasted state. Under the guidelines of the U.S. Food and Drug Administration, two products or methods are bioequivalent if the 90% confidence intervals for AUC and Cₘₐₓ are between 0.80 and 1.25. Under the guidelines of the European Medicines Agency (EMEA), two products or methods are bioequivalent if the 90% confidence interval for active compound is between 0.80 and 1.25 and the 90% confidence interval for Cₘₐₓ is between 0.70 and 1.43.

In various embodiments of the present invention, the formulation is one in which, within 5 minutes following administration, at least about 20%, about 30%, or about 40% of the active compound is dissolved. In various embodiments of the present invention, the formulation is one in which, within 10 minutes following administration, at least about 40%, about 50%, about 60%, about 70%, or about 80% is dissolved. In various embodiments of the present invention, the formulation is one in which, within 20 minutes following administration, at least about 70%, about 80%, about 90%, or about 100% of the active compound is dissolved. Dissolution is preferably measured in a medium which is predictive of in vivo dissolution of a composition, for example, an aqueous medium containing 0.025M sodium lauryl sulfate. Determination of the amount dissolved can be carried out by spectrophotometry. The rotating blade method (European Pharmacopoeia) may also be used to measure dissolution.

Upon administration of a formulation containing nanoparticles to a subject, the nanoparticles therein may redisperse in vivo. In an embodiment of the present invention, the nanoparticles in the formulation redisperse, following administration thereof to a subject, such that the effective average particle size of the particles is less than about 2000 nm, as measured by appropriate methods, for example, light-scattering methods and microscopy. In various other embodiments of the present invention, the redispersed nanoparticles have an effective average particle size of less than about 1900 nm, about 1800 nm, about 1700 nm, about 1600 nm, about 1500 nm, about 1400 nm, about 1300 nm, about 1200 nm, about 1100 nm, about 1000 nm, about 900 nm, about 800 nm, about 700 nm, about 600 nm, about 500 nm, about 400 nm, about 300 nm, about 250 nm, about 200 nm, about 150 nm, about 100 nm, about 75 nm, or about 50 nm, as measured by appropriate methods, for example, light-scattering methods and microscopy.

Whether a formulation exhibits the above property may be demonstrated by whether it exhibits this property in biorelevant aqueous media. Such biorelevant aqueous media may be any aqueous media that exhibits ionic strength and pH that are representative of physiological conditions found in the human body. Such media can be, for example, aqueous electrolyte solutions of aqueous solutions of any salt, acid, or base, or a combination thereof, which exhibits the desired pH and ionic strength. Biorelevant pH is well known in the art. For example, in the stomach, the pH ranges from slightly less than 2 (but typically greater than 1) up to 4 or 5. In the small intestine, the pH can range from 4 to 6. In the colon, the pH can range from 6 to 8. Biorelevant ionic strength is also well known in the art. Fasted state gastric fluid has an ionic strength of about 0.1M while fasted state intestinal fluid has an ionic strength of about 0.14M. Appropriate pH and ionic strength values can be obtained through numerous combinations of acids, bases, salts, etc.

The nanoparticles comprising the active compound may be made by various methods. Examples of such methods include milling, homogenization, precipitation, freezing, template emulsion techniques, or any combination thereof.

In the milling method, particles comprising an active compound may be dispersed in a liquid dispersion medium in which the active compound is poorly soluble (e.g., water, safflower oil, ethanol, t-butanol, glycerin, polyethylene glycol (PEG), hexane, glycol). This may then be followed by the application of a mechanical means to reduce the size of the particles to the desired effective average particle size. The active-containing particles may be reduced in size in the presence of the surface modifier or the particles may be contacted with the surface modifier prior to or following size reduction.

In the microprecipitation method, the active compound may be dissolved in a suitable solvent and the resulting composition is added to a solution comprising the surface modifier. The resulting active-containing nanoparticles may then be precipitated from the solution using an appropriate non-solvent. Any formed salt may be removed by dialysis or diafiltration and concentration of the dispersion by conventional means.

In the homogenization method, active-containing particles may be dispersed in a first dispersion medium. This dispersion may then be subjected to homogenization to reduce the size of the particles to the desired effective average particle size. Such reduction may take place in the presence of a surface modifier or, alternatively, the modifier may be contacted with the particles prior to or following size reduction.

The formation of nanoparticles by freezing may be accomplished by, for example, spray freezing into liquid (SFL) or ultra rapid freezing (URF). In the spray freezing into liquid (SFL) method, an organic or organoaqueous solution comprising the active compound and a surface modifier is injected into a cryogenic liquid (e.g., liquid nitrogen). Droplets of the solution then freeze at a rate sufficient to minimize crystallization and particle growth, thus forming the desired nanoparticles comprising the active compound and the surface modifier. In the ultra rapid freezing (URF) method, a water-miscible, anhydrous, organic, or organoaqueous solution of the active compound and the surface modifier is applied onto a cryogenic substrate. The solvent is then removed by means such as lyophilization or atmospheric freeze-drying with the resulting nanostructured particles remaining.

In the template emulsion method, an oil-in-water emulsion is prepared and then swelled with a non-aqueous solution comprising an active compound and a surface modifier. The solvent and water are then removed and stabilized nanoparticles are recovered. The size of the particles formed is a direct result of the size of the emulsion droplets prior to the loading thereof with the active compound-containing solution. Accordingly, this property can be controlled and optimized. In addition, the stability of the emulsion can be adjusted by the choice of solvents and surface modifiers.

The formulation of the present invention may comprise also one or more binding agents, filling agents, lubricating agents, suspending agents, sweeteners, flavoring agents, preservatives, buffers, wetting agents, disintegrants, effervescent agents, anti-adherents, and other excipients. Such excipients are known in the art. In embodiments of the present invention which involve the use of particles, including nanoparticles, these excipients may be present within the particle.

Examples of binding agents include hydroxypropylmethylcellulose (HPMC).

Examples of filling agents are lactose monohydrate, lactose anhydrous, and various starches.

Examples of binding agents are various celluloses and cross-linked polyvinylpyrrolidone, microcrystalline cellulose, such as Avicel® PH101 and Avicel® PH102, microcrystalline cellulose, and silicified microcrystalline cellulose (ProSolv SMCCTM).

Suitable lubricants, including agents that act on the flowability of the powder to be compressed, are colloidal silicon dioxide, such as Aerosil® 200, talc, stearic acid, magnesium stearate, calcium stearate, and silica gel.

Examples of sweeteners are any natural or artificial sweetener, such as sucrose, xylitol, sodium saccharin, cyclamate, aspartame, and acsulfame. Examples of flavoring agents are Magnasweet® (trademark of MAFCO), bubble gum flavor, and fruit flavors, and the like.

Examples of preservatives are potassium sorbate, methylparaben, propylparaben, benzoic acid and its salts, other esters of parahydroxybenzoic acid such as butylparaben, alcohols such as ethyl or benzyl alcohol, phenolic compounds such as phenol, or quarternary compounds such as benzalkonium chloride.

Suitable diluents include pharmaceutically acceptable inert fillers, such as microcrystalline cellulose, lactose, dibasic calcium phosphate, saccharides, and/or mixtures of any of the foregoing. Examples of diluents include microcrystalline cellulose, such as Avicel® PH101 and Avicel® PH102; lactose such as lactose monohydrate, lactose anhydrous, and Pharmatose® DCL21; dibasic calcium phosphate such as Emcompress®; mannitol; starch; sorbitol; sucrose; and glucose.

Suitable disintegrants include lightly crosslinked polyvinyl pyrrolidone, corn starch, potato starch, maize starch, and modified starches, croscarmellose sodium, cross-povidone, sodium starch glycolate, and mixtures thereof.

Examples of effervescent agents are effervescent couples such as an organic acid and a carbonate or bicarbonate. Suitable organic acids include, for example, citric, tartaric, malic, fumaric, adipic, succinic, and alginic acids and anhydrides and acid salts. Suitable carbonates and bicarbonates include, for example, sodium carbonate, sodium bicarbonate, potassium carbonate, potassium bicarbonate, magnesium carbonate, sodium glycine carbonate, L-lysine carbonate, and arginine carbonate. Alternatively, only the sodium bicarbonate component of the effervescent couple may be present.

Examples of anti-adherents include silicon dioxide and talc.

In an embodiment of the present invention, the narcotic analgesic and/or the non-narcotic analgesic may be in a particulate dosage form. The particle may be in the form of spheres, for example, microspheres, pellets, beads, or granules. The particle may contain the narcotic analgesic alone, the non-narcotic analgesic alone, or both the narcotic analgesic and the non-narcotic analgesic. In an embodiment in which the narcotic analgesic and/or non-narcotic analgesic is contained in a nanoparticle, the particle of the dosage form may be a nanoparticle. Alternatively, the particle may contain nanoparticles which comprise the narcotic and/or the non-narcotic analgesic. A formulation comprising multiple particles is termed a “multiparticulate” formulation.

In an embodiment of the present invention, the aforementioned particle is an “immediate release particle”. By “immediate release”, it is meant that the particle releases a compound therein immediately upon dissolution of the particle.

In an embodiment of the present invention, the particle is a modified release particle. By “modified release”, it is meant that the particle allows for a release of a compound from the particle that is not immediate. For example, the release may be controlled or it may be delayed. By “controlled release” it is meant that the release of the compound is characterized by a specific release profile in which, for a specific period of time, a specific rate of release is achieved. Various different rates of release may be achieved at different periods of time. By “delayed release” it is meant that the compound is released after a period of delay in which the compound is not released. The compound may be released immediately following the period of delay, in which case the particle is considered to be a “delayed immediate release” particle. Alternatively, the compound may be released on a controlled release basis following the initial delay period, in which case the particle is considered to be a “delayed controlled release” particle.

In an embodiment of the present invention, a compound of interest (e.g., a narcotic analgesic, a non-narcotic analgesic) is released from the formulation in a “pulsatile” manner. A pulsatile release profile is one in which, over the course of time, at least two periods in which there are relatively high blood plasma concentrations of the compound (“peaks”) are separated by a period of relatively low blood plasma concentration level of the compound (a “trough”). Pulsatile release profiles in which there are two peaks are called “bimodal” release profiles. A bimodal release profile may be achieved, for example, by the combination of particles which allow for the immediate release of the compound of interest with particles which allow for the delayed release of the compound after a period of time. Additional populations containing particles which allow for the delayed release of the compound after differing periods of time may be used to create a release profile with additional higher blood plasma concentration “peaks”.

In another embodiment, a compound of interest (e.g., a narcotic analgesic, a non-narcotic analgesic) is released from the formulation in a “continuous” manner. In such a release, the compound of interest is released in continuously, either at a constant or a variable rate. This may be achieved by the use of modified release particles, including two or more different populations of modified release particles with each population releasing the compound of interest at different rates.

To allow for modified release of the compound of interest (for example, a narcotic analgesic or a non-narcotic analgesic), the particle may contain a modified release coating or a modified release matrix. The coating or matrix serves to retard the release of the compound from the particle. The release characteristics of a particle may be adjusted by adjusting the amount of the coating or matrix, for example, by applying a thicker coating to the particle, or by adjusting the ingredients of the coating or matrix.

Any coating material which modifies the release of the compound of interest (a narcotic or a non-narcotic analgesic) in the desired manner may be used. Examples of coating materials which are suitable for use in the practice of the present invention include: polymer coating materials, such as cellulose acetate phthalate, cellulose acetate trimaletate, hydroxy propyl methylcellulose phthalate, polyvinyl acetate phthalate, ammonio methacrylate copolymers such as those sold under the trademark Eudragit® RS and RL, poly acrylic acid and poly acrylate and methacrylate copolymers such as those sold under the trademark Eudragit® S and L, polyvinyl acetaldiethylamino acetate, hydroxypropyl methylcellulose acetate succinate, and shellac; hydrogels and gel-forming materials, such as carboxyvinyl polymers, sodium alginate, sodium carmellose, calcium carmellose, sodium carboxymethyl starch, poly vinyl alcohol, hydroxyethyl cellulose, methyl cellulose, gelatin, starch, and cellulose based cross-linked polymers—in which the degree of crosslinking is low so as to facilitate adsorption of water and expansion of the polymer matrix, hydroxypropyl cellulose, hydroxypropylmethylcellulose (HPMC), polyvinylpyrrolidone, crosslinked starch, microcrystalline cellulose, chitin, aminoacryl-methacrylate copolymer (Eudragit® RS-PM, Rohm & Haas), pullulan, collagen, casein, agar, gum arabic, sodium carboxymethyl cellulose, (swellable hydrophilic polymers) poly(hydroxyalkyl methacrylate), polyvinylpyrrolidone, anionic and cationic hydrogels, polyvinyl alcohol having a low acetate residual, a swellable mixture of agar and carboxymethyl cellulose, copolymers of maleic anhydride and styrene, ethylene, propylene or isobutylene, pectin (m. wt. about 30 k-300 k), polysaccharides such as agar, acacia, karaya, tragacanth, algins and guar, polyacrylamides, AquaKeep® acrylate polymers, diesters of polyglucan, crosslinked polyvinyl alcohol and poly N-vinyl-2-pyrrolidone, sodium starch glucolate; hydrophilic polymers such as polysaccharides, methyl cellulose, sodium or calcium carboxymethyl cellulose, nitro cellulose, carboxymethyl cellulose, cellulose ethers, polyethylene oxides (e.g. Polyox®, Union Carbide), methyl ethyl cellulose, ethylhydroxy ethylcellulose, cellulose acetate, cellulose butyrate, cellulose propionate, gelatin, collagen, starch, maltodextrin, pullulan, polyvinyl pyrrolidone, polyvinyl alcohol, polyvinyl acetate, glycerol fatty acid esters, polyacrylamide, polyacrylic acid, copolymers of methacrylic acid or methacrylic acid (e.g. Eudragit®, Rohm and Haas), other acrylic acid derivatives, sorbitan esters, natural gums, lecithins, pectin, alginates, ammonia alginate, sodium, calcium, potassium alginates, propylene glycol alginate, agar, and gums such as arabic, karaya, locust bean, tragacanth, carrageens, guar, xanthan, scleroglucan and mixtures and blends thereof.

As will be appreciated by the person skilled in the art, excipients such as plasticisers, lubricants, solvents and the like may be added to the coating. Suitable plasticisers include for example acetylated monoglycerides; butyl phthalyl butyl glycolate; dibutyl tartrate; diethyl phthalate; dimethyl phthalate; ethyl phthalyl ethyl glycolate; glycerin; propylene glycol; triacetin; citrate; tripropioin; diacetin; dibutyl phthalate; acetyl monoglyceride; polyethylene glycols; castor oil; triethyl citrate; polyhydric alcohols, glycerol, acetate esters, gylcerol triacetate, acetyl triethyl citrate, dibenzyl phthalate, dihexyl phthalate, butyl octyl phthalate, diisononyl phthalate, butyl octyl phthalate, dioctyl azelate, epoxidised tallate, triisoctyl trimellitate, diethylhexyl phthalate, di-n-octyl phthalate, di-i-octyl phthalate, di-i-decyl phthalate, di-n-undecyl phthalate, di-n-tridecyl phthalate, tri-2-ethylhexyl trimellitate, di-2-ethylhexyl adipate, di-2-ethylhexyl sebacate, di-2-ethylhexyl azelate, dibutyl sebacate. Suitable solvents include acetone and isopropyl alcohol.

In an embodiment in which a delayed immediate release is desired, the coating used may be enteric. Enteric coatings comprise pH sensitive polymers. Typically, these polymers are carboxylated and interact sparingly with water at low pH. However, at a high pH, the polymer ionizes which causes swelling or the dissolution of the polymers. Such coatings may, therefore, remain intact in the acidic environment of the stomach and then dissolve in the more alkaline environment of the intestine.

Any matrix material which modifies the release of the compound of interest (a narcotic or a non-narcotic analgesic) in the desired manner may be used. Examples of matrix materials which are suitable for use in the practice of the present invention include: hydrophilic polymers, hydrophobic polymers and mixtures thereof which are capable of modifying the release of the compound of interestdispersed therein in vitro or in vivo: Modified-release matrix materials suitable for the practice of the present invention include but are not limited to microcrytalline cellulose, sodium carboxymethylcellulose, hydroxyalkylcelluloses such as hydroxypropylmethylcellulose (HPMC) and hydroxypropylcellulose, polyethylene oxide, alkylcelluloses such as methylcellulose and ethylcellulose, polyethylene glycol, polyvinylpyrrolidone, cellulose acetate, cellulose acetate butyrate, cellulose acetate phthalate, cellulose acetate trimellitate, polyvinylacetate phthalate, polyalkylmethacrylates, polyvinyl acetate and mixture thereof.

In an embodiment of the invention, the formulation releases the narcotic analgesic and the non-narcotic analgesic in such a manner that the duration of action of the narcotic analgesic matches that of the non-narcotic analgesic. This may be accomplished by, for example, using modified release particles which comprise the narcotic analgesic and/or modified release particles which comprise the non-narcotic analgesic. The release is modified such that the release of one active compound is over a period of time such that the duration of action of that compound matches that of the other active compound. In such an embodiment, the release of the second active compound may also be modified.

An immediate release particle may be made, for example, by coating a solution comprising the compound of interest onto an inert bead (for example, a sugar sphere). Following coating, the solvent dries off, leaving the immediate release particle.

A modified release particle may be made, for example, by coating an immediate release particle such as that described above with a solution comprising the compounds of a modified release coating. Following coating, the solvent dries off, leaving the modified release particle.

The particles described above may be combined to form a larger solid dosage form, for example a tablet, a capsule, a lozenge, etc.

The invention provides a method for the treatment of pain comprising the step of delivering to the patient a formulation comprising a narcotic analgesic and a non-narcotic analgesic.

The formulation may be administered to a subject via any conventional means including, but not limited to, orally, rectally, ocularly, parenterally (e.g., intravenous, intramuscular, or subcutaneous), intracistemally, pulmonary, intravaginally, intraperitoneally, locally (e.g., powders, ointments or drops), or as a buccal or nasal spray.

Compositions suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carders, diluents, solvents, or vehicles including water, ethanol, polyols (propyleneglycol, polyethylene glycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions, and by the use of surfactants.

The formulations of the present invention may be made by methods known in the art for mixing a narcotic analgesic and a non-narcotic analgesic. For example, particles comprising a narcotic analgesic may be encapsulated with particles comprising a non-narcotic analgesic.

### EXAMPLE 1

This example describes the preparation of immediate release particles comprising a narcotic analgesic.

Solutions comprising a narcotic analgesic (hydrocodone) are prepared ((A) to (F)). The formulations of these solutions is shown in Table 1.

TABLE 1

| | Narcotic Analgesic Solutions for | | | | | |
|---|---|---|---|---|---|---|
| | Immediate Release Particles | | | | | |
| | (A) | (B) | (C) | (D) | (E) | (F) |
| Ingredient | Amount (percent by weight) | | | | | |
| Hydrocodone | 6.0 | 6.0 | 6.0 | 6.0 | 6.0 | 6.0 |
| HPMC 2910 | 1.0 | 2.0 | 2.0 | — | — | 1.5 |
| PEG 6000 | — | — | — | 0.5 | — | — |
| Povidone K30 | — | — | — | — | 5.0 | — |
| Fumaric Acid | — | 6.0 | — | — | — | — |
| Citric Acid | — | — | 6.0 | — | — | — |
| Silicon Dioxide | 1.5 | 1.0 | 1.0 | — | — | 2.0 |
| Talc | 1.5 | — | — | — | — | — |
| Purified Water | 90.0 | 85.0 | 85.0 | 93.5 | 89.0 | 90.5 |

Each of these solutions is then coated onto inert sugar spheres (30/35 mesh). The resulting particles have a mean diameter of 0.5 to 0.6 mm.

Hydroxypropylmethylcellulose (HPMC) acts as a binding agent for this coating. Silicon dioxide is an anti-adherent.

### EXAMPLE 2

This example describes the preparation of immediate release particles comprising a non-narcotic analgesic.

Solutions comprising a non-narcotic analgesic (aspirin) are prepared ((A) to (F)). The formulations of these solutions is shown in Table 2.

TABLE 2

| | Non-narcotic Analgesic Solutions for | | | | | |
|---|---|---|---|---|---|---|
| | Immediate Release Particles | | | | | |
| | (A) | (B) | (C) | (D) | (E) | (F) |
| Ingredient | Amount (percent by weight) | | | | | |
| Aspirin | 6.0 | 6.0 | 6.0 | 6.0 | 6.0 | 6.0 |
| HPMC 2910 | 1.0 | 2.0 | 2.0 | — | — | 1.5 |
| PEG 6000 | — | — | — | 0.5 | — | — |
| Povidone K30 | — | — | — | — | 5.0 | — |
| Fumaric Acid | — | 6.0 | — | — | — | — |
| Citric Acid | — | — | 6.0 | — | — | — |
| Silicon Dioxide | 1.5 | 1.0 | 1.0 | — | — | 2.0 |
| Talc | 1.5 | — | — | — | — | — |
| Purified Water | 90.0 | 85.0 | 85.0 | 93.5 | 89.0 | 90.5 |

Each of these solutions is then coated onto inert sugar spheres (30/35 mesh). The resulting particles have a mean diameter of 0.5 to 0.6 mm. Hydroxypropylmethylcellulose (HPMC) acts as a binding agent for this coating. Silicon dioxide is an anti-adherent.

### EXAMPLE 3

This example describes the preparation of modified release particles comprising a narcotic analgesic.

Immediate release particles comprising a narcotic analgesic (hydrocodone), such as those prepared in Example 1, are coated with a solution which forms a modified release coating around the particle. Examples of such solutions are provided in Table 3 ((A) to (G)).

TABLE 3

| | Non-narcotic Analgesic Solutions for | | | | | | |
|---|---|---|---|---|---|---|---|
| | Immediate Release Particles | | | | | | |
| | (A) | (B) | (C) | (D) | (E) | (F) | (G) |
| Ingredient | Amount (percent by weight) | | | | | | |
| Eudragit ® RS 100 | 4.1 | 4.9 | 5.5 | 4.4 | — | 5.5 | 7.5 |
| Eudragit ® RL 100 | — | 0.5 | — | 1.1 | — | — | — |
| Eudragit ® L 100 | 1.4 | — | — | — | — | — | — |
| Ethocel | — | — | — | — | 3.0 | — | — |
| Triethyl Citrate | 1.5 | 1.6 | — | 1.1 | — | — | 1.5 |
| Dibutyl Sebacate | — | — | — | — | 0.6 | 1.0 | — |
| Silicon Dioxide | 1.0 | 1.0 | 1.0 | — | 2.0 | 1.0 | — |
| Talc | 2.5 | 2.5 | 1.0 | 2.8 | — | 1.0 | 2.5 |
| Acetone | 34.0 | 34.0 | 15.0 | 35.6 | — | 14.0 | 33.5 |
| Isopropyl Alcohol | 50.0 | 50.0 | 72.5 | 50.0 | 94.4 | 72.5 | 50.0 |
| Purified Water | 5.5 | 5.5 | 5.0 | 5.0 | — | 5.0 | 5.0 |

Ammonio methacrylate copolymer (Eudragit® RS 100) is a rate-controlling polymer which imparts the controlled-release properties to the particles. Talc is used as an anti-adherent. Acetone and isopropyl alcohol are solvents used in forming a solution of the ammonio methacrylate copolymer. Following the coating of the solution onto the immediate release particle, the solvents evaporate, thus forming a solid coating around the particle. The resulting coated particles are then dried in a oven for 10 to 20 hours at 40 to 500° C./30 to 60% RH to remove any residual solvents and to obtain a moisture content of about 3 to 6%.

### EXAMPLE 4

This example describes the preparation of modified release particles comprising a non-narcotic analgesic.

Immediate release particles comprising a non-narcotic analgesic (aspirin), such as those prepared in Example 2, are coated with a solution which forms a modified release coating around the particle. Examples of such solutions are provided in Table 4 ((A) to (G)).

TABLE 4

| | Modified Release Solutions | | | | | | |
|---|---|---|---|---|---|---|---|
| | (A) | (B) | (C) | (D) | (E) | (F) | (G) |
| Ingredient | Amount (percent by weight) | | | | | | |
| Eudragit ® RS 100 | 4.1 | 4.9 | 5.5 | 4.4 | — | 5.5 | 7.5 |
| Eudragit ® RL 100 | — | 0.5 | — | 1.1 | — | — | — |
| Eudragit ® L 100 | 1.4 | — | — | — | — | — | — |
| Ethocel | — | — | — | — | 3.0 | — | — |
| Triethyl Citrate | 1.5 | 1.6 | — | 1.1 | — | — | 1.5 |
| Dibutyl Sebacate | — | — | — | — | 0.6 | 1.0 | — |
| Silicon Dioxide | 1.0 | 1.0 | 1.0 | — | 2.0 | 1.0 | — |
| Talc | 2.5 | 2.5 | 1.0 | 2.8 | — | 1.0 | 2.5 |
| Acetone | 34.0 | 34.0 | 15.0 | 35.6 | — | 14.0 | 33.5 |
| Isopropyl Alcohol | 50.0 | 50.0 | 72.5 | 50.0 | 94.4 | 72.5 | 50.0 |
| Purified Water | 5.5 | 5.5 | 5.0 | 5.0 | — | 5.0 | 5.0 |

Ammonio methacrylate copolymer (Eudragit® RS 100) is a rate-controlling polymer which imparts the controlled-release properties to the particles. Talc is used as an anti-adherent. Acetone and isopropyl alcohol are solvents used in forming a solution of the ammonio methacrylate copolymer. Following the coating of the solution onto the immediate release particle, the solvents evaporate, thus forming a solid coating around the particle. The resulting coated particles are then dried in a oven for 10 to 20 hours at 40 to 500° C./30 to 60% RH to remove any residual solvents and to obtain a moisture content of about 3 to 6%.

### EXAMPLE 5

This example describes the preparation of nanoparticles comprising a narcotic analgesic (hydrocodone).

Thirty grams of hydroxypropylcellulose (Klucel Type EF; Aqualon) is dissolved in 670 grams of deionized water using a continuous laboratory mixer. The hydroxypropylcellulose serves as a surface modifier. Three hundred grams of hydrocodone is then dispersed into the solution until a homogenous suspension is obtained. A laboratory scale media mill filled with polymeric grinding media is used in a continuous fashion until the mean particle size is approximately 200 nm as measured using a laser light scattering technique.

### EXAMPLE 6

This example also describes the preparation of nanoparticles comprising a narcotic analgesic (hydrocodone).

Twenty five grams of polyvinylpyrrolidone (K29/32; BASF Corpl) is dissolved in 575 grams of deionized water using a continuous laboratory mixer. The polyvinylpyrrolidone serves as a surface modifier. Four hundred grams of hydrocodone is then dispersed into the solution until a homogenous suspension is obtained. A laboratory scale media mill filled with polymeric grinding media is used in a continuous fashion until the mean particle size is approximately 200 nm as measured using a laser light scattering technique.

### EXAMPLE 7

This example describes the preparation of nanoparticles comprising a non-narcotic analgesic (aspirin).

Thirty grams of hydroxypropylcellulose (Klucel Type EF; Aqualon) is dissolved in 670 grams of deionized water using a continuous laboratory mixer. The hydroxypropylcellulose serves as a surface modifier. Three hundred grams of aspirin is then dispersed into the solution until a homogenous suspension is obtained. A laboratory scale media mill filled with polymeric grinding media is used in a continuous fashion until the mean particle size is approximately 200 nm as measured using a laser light scattering technique.

### EXAMPLE 8

This example also describes the preparation of nanoparticles comprising a non-narcotic analgesic (aspirin).

Twenty five grams of polyvinylpyrrolidone (K29/32; BASF Corpl) is dissolved in 575 grams of deionized water using a continuous laboratory mixer. The polyvinylpyrrolidone serves as a surface modifier. Four hundred grams of aspirin is then dispersed into the solution until a homogenous suspension is obtained. A laboratory scale media mill filled with polymeric grinding media is used in a continuous fashion until the mean particle size is approximately 200 nm as measured using a laser light scattering technique.

What is claimed is:

1. A formulation comprising a narcotic analgesic and a non-narcotic analgesic.

2. A formulation according to claim 1 wherein said narcotic analgesic is contained in particles and said non-narcotic analgesic is separately contained in separate particles.

3. A formulation according to claim 2 wherein said particles are modified release particles.

4. A formulation according to claim 3 wherein said particles comprising a non-narcotic analgesic release said non-narcotic analgesic such that the duration of action of said non-narcotic analgesic matches that of said narcotic analgesic.

5. A formulation according to claim 4 wherein said particles comprising a non-narcotic analgesic are modified release particles.

6. A formulation according to claim 3 wherein said particles comprising a narcotic analgesic release said narcotic analgesic such that the duration of action of said narcotic analgesic matches that of said non-narcotic analgesic.

7. A formulation according to claim 6 wherein said particles comprising a narcotic analgesic are modified release particles.

8. A method for the treatment of pain comprising administering a therapeutically effective amount of a formulation according to claim 1.

9. A method for preparing a formulation which is useful in the treatment of pain comprising the step of mixing a narcotic analgesic and a non-narcotic analgesic.
